(19) United States
(12) Patent Application Publication
(10) Pub. No.: US 2026/0283854 A1
(43) Pub. Date: Sep. 24, 2026

(54) BINDER COMPRESSION AID

(71) Applicant: Ann Marie Hayek, Oldsmar, FL (US)

(72) Inventor: Ann Marie Hayek, Oldsmar, FL (US)

(21) Appl. No.: 19/086,034

(22) Filed: Mar. 20, 2025

(51) Int. Cl. A61F 13/00

(52) U.S. Cl. CPC A61F 13/00051

(57) ABSTRACT

A binder compression aid may comprise a bracket configured to be mounted to a fixed surface. The bracket may include a mounting section for attaching to the fixed surface and a protruding section extending from the mounting section. A slot in the protruding section may be configured to receive an abdominal binder. A securing mechanism on the protruding section may be configured to temporarily hold one end of the abdominal binder. The securing mechanism may comprise a strip of hook-and-loop fastener material. At least one attachment mechanism may be provided for mounting the bracket to the fixed surface. The attachment mechanism may comprise suction cups or strap slots configured to receive straps for securing the bracket. The mounting section and protruding section may be perpendicular to each other.

### FIELD OF THE INVENTION

The present disclosure relates generally to medical devices, and more particularly to devices and methods for assisting in the application of compression binders for the body.

### BACKGROUND

Binders are compression garments commonly used after various surgeries to the abdomen, back, chest or torso to provide support, aid in recovery and help in body contouring. Typically, these binders need to be wrapped tightly around a patient's torso or chest and back, which can be difficult for a patient to accomplish alone, especially in the early stages of recovery when mobility may be limited.

Conventional methods often require assistance from another person to properly apply the binder. This dependence on others can be inconvenient and may limit a patient's independence during recovery. There exists a need for a device that enables patients to independently and effectively apply their compression binders.

### SUMMARY

The present disclosure is directed to a device and method for assisting in the self-application of a compression binder. In one aspect, a binder compression aid may include a bracket configured to be mounted to a fixed surface. The bracket may have a slot for passing an abdominal binder through and a securing mechanism for temporarily holding one end of the binder. In some embodiments, the bracket may be an angled bracket with two perpendicular sections—a mounting section for attaching to a surface and a protruding section with the slot and securing mechanism. The securing mechanism may include a strip of hook-and-loop fastener material along the length of the slot. This allows for easy attachment and detachment of the abdominal binder. Various attachment mechanisms may be provided for mounting the bracket to different surfaces. These may include suction cups for any non-porous surfaces, straps for door frames or posts, and options for permanent mounting such as screws or nails.

A method for self-applying a binder using the compression aid may include mounting the bracket at an appropriate height, threading one end of the binder through the slot and securing it to the bracket, stretching the binder out and wrapping it around the body while maintaining tension, and finally detaching the binder from the bracket to complete the application. The binder compression aid allows users to independently apply their binders with proper tension, potentially improving consistency of use and effectiveness of the binder during recovery.

### BRIEF DESCRIPTION OF THE DRAWINGS

The accompanying drawings, which are incorporated in and constitute a part of this specification, illustrate embodiments of the disclosure and, together with the description, serve to explain the principles of the disclosure.

FIG. 1 is a perspective view of a binder compression aid according to an embodiment of the present disclosure.

FIG. 2 shows the side and top views of a strap bracket component of the binder compression aid according to an embodiment of the present disclosure.

FIG. 3 is a side view of the binder compression aid mounted on a glass door according to an embodiment of the present disclosure.

FIG. 4 is a front view of the binder compression aid mounted on a surface according to an embodiment of the present disclosure.

FIG. 5 is another side view of the binder compression aid mounted on a glass door according to an embodiment of the present disclosure.

FIG. 6 is a perspective view showing a hand gripping the abdominal binder and threading one end of abdominal binder through wrap pass thru slot on the compression aid mounted on a door according to an embodiment of the present disclosure.

FIG. 7 is a perspective view showing the abdominal binder securing the end of abdominal biner to fastener of compression aid compression aid mounted on a sliding door according to an embodiment of the present disclosure.

FIG. 8 is a flowchart illustrating an example method for implementing the binder compression aid according to various embodiments of the present disclosure.

FIG. 9 shows a method of using the binder compression aid utilizing a door frame.

FIG. 10A and FIG. 10B 9 shows a method of using the binder compression aid utilizing a post.

### DETAILED DESCRIPTION

Reference will now be made in detail to exemplary embodiments of the present disclosure, examples of which are illustrated in the accompanying drawings. Wherever possible, the same reference numbers will be used throughout the drawings to refer to the same or like parts. The present disclosure provides devices and methods for assisting in the self-application of compression binders. While the devices and methods described herein are particularly useful for post-surgical patients, it should be understood that they may be used in various other contexts where compression or support is desired. For example, chest binders are garments designed to compress and flatten the appearance of the chest. They are commonly used by anyone who desires a flatter chest for personal or aesthetic reasons or align their physical appearance with their gender identity. Binders are also used by performers or athletes, to achieve a streamlined look.

It should be appreciated that this description is for illustrative purposes only and is not intended to limit the scope of the present disclosure. In addition, various features are described herein in a certain order or arrangement, but such features may be combined, separated, or rearranged without departing from the spirit and scope of the disclosure. In other instances, well-known features have not been described in detail to avoid unnecessarily complicating the description.

Throughout this specification and the claims, the terms “comprise,” “comprising,” “include,” “including,” and the like are to be understood to imply the inclusion of stated elements but not the exclusion of any other elements. The term “exemplary” is used in the sense of “example” rather than “ideal” or “model.” As used herein, the terms “about” or “approximately” apply to all numeric values, whether or not explicitly indicated. These terms generally refer to a range of numbers that one of skill in the art would consider equivalent to the recited values (i.e., having the same function or result).

Before the present articles, systems, apparatuses, and/or methods are disclosed and described, it is to be understood that they are not limited to specific methods unless otherwise specified, or to particular materials unless otherwise specified, as such can, of course, vary. It is also to be understood that the terminology used herein is for the purpose of describing particular aspects only and is not intended to be limiting. Although any methods and materials similar or equivalent to those described herein can be used in the practice or testing of the present disclosure, example methods and materials are now described.

### A. Definitions

It is also to be understood that the terminology used herein is for the purpose of describing particular aspects only and is not intended to be limiting. As used in the specification and in the claims, the term “comprising” can include the aspects “consisting of” and “consisting essentially of” Unless defined otherwise, all technical and scientific terms used herein have the same meaning as commonly understood by one of ordinary skill in the art to which this disclosure belongs. In this specification and in the claims which follow, reference will be made to a number of terms which shall be defined herein.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

The terms “first,” “second,” “first part,” “second part,” and the like, where used herein, do not denote any order, quantity, or importance, and are used to distinguish one element from another, unless specifically stated otherwise. As used herein, the terms “optional” or “optionally” means that the subsequently described event or circumstance can or cannot occur, and that the description includes instances where said event or circumstance occurs and instances where it does not. For example, the phrase “optionally affixed to the surface” means that it can or cannot be fixed to a surface.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

It is understood that the apparatuses and systems disclosed herein have certain functions. Disclosed herein are certain structural requirements for performing the disclosed functions, and it is understood that there are a variety of structures that can perform the same function that are related to the disclosed structures, and that these structures will typically achieve the same result. The following description of various embodiments is merely exemplary in nature and is in no way intended to limit the disclosure, its application, or uses.

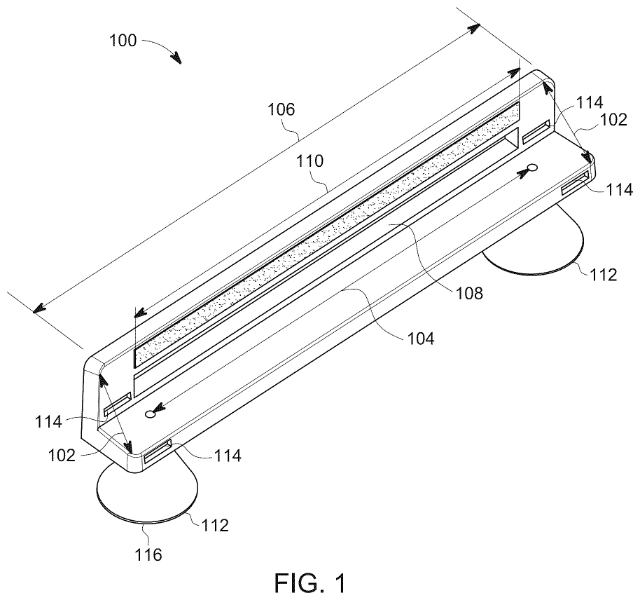

Referring to FIG. 1, a binder compression aid 100 is shown according to an embodiment of the present disclosure. The compression aid 100 may include an angled bracket 102 having two perpendicular sections: a mounting section 104 and a protruding section 106. The mounting section 104 may be configured to attach to a fixed surface, while the protruding section 106 may include features for interacting with an abdominal binder. The protruding section 106 may include a slot 108, referred to as a “wrap pass thru slot” in FIG. 1, through which an abdominal binder may be passed. Above the slot 108, a strip of hook-and-loop fastener material 110 (e.g., Velcro) may be provided. This fastener material 110 may serve as a securing mechanism for temporarily holding one end of the abdominal binder during the application process.

The compression aid 100 may include various attachment mechanisms for securing it to different surfaces. In the embodiment shown in FIG. 1, two suction cups 112 are provided at the top and bottom of the mounting section 104. These suction cups 112 may be particularly useful for attaching the compression aid 100 to any non-porous surface such as heavy glass doors or marble. Additionally, the compression aid 100 may include strap slots 114 at each end of the mounting section 104. These strap slots 114 may allow for alternative mounting options, such as using straps to secure the compression aid 100 to a door frame, bedpost, or other suitable structure.

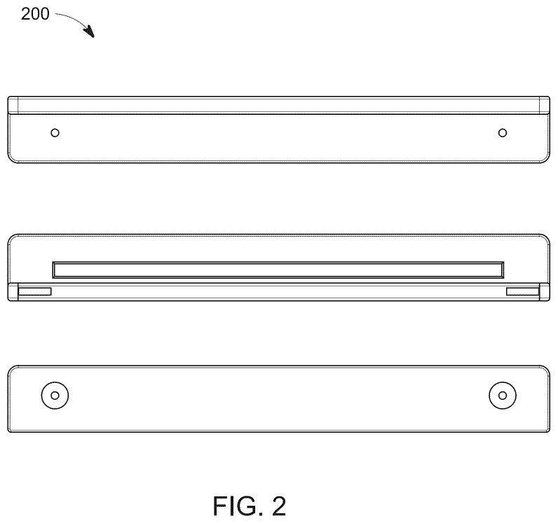

FIG. 2 provides an illustration for the side, top and of the strap bracket component of the compression aid. As shown, the bracket may have dimensions of approximately 15 to 20 inches in length for each section, with a width of about 2.5 inches to possibly 3 inches. The wrap pass thru slot may be approximately 12.5 INCHES TO 16.5 inches long and 0.5 inches wide. These dimensions may be adjusted based on the specific needs of the user and the typical dimensions of abdominal binders to be used with the device.

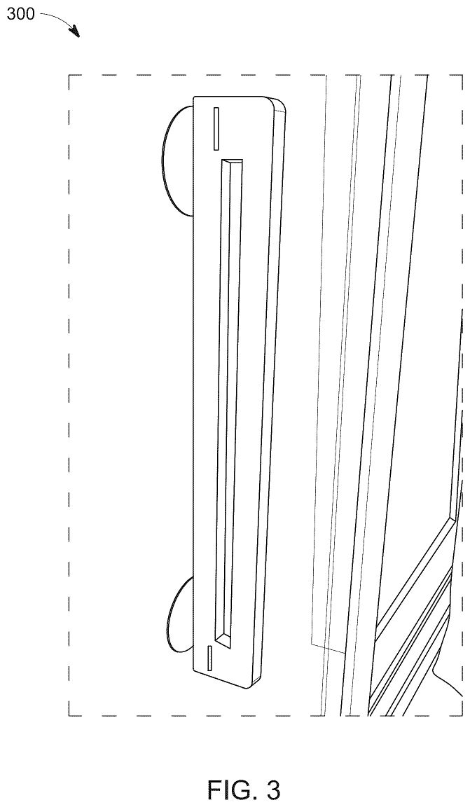

Referring now to FIGS. 3-5, various views of the compression aid 100 mounted on a glass door are shown. These figures illustrate how the suction cups 112 may be used to securely attach the compression aid 100 to a smooth non-porous vertical surface. The protruding section 106 extends outward from the mounting surface, providing easy access for threading and securing the abdominal binder.

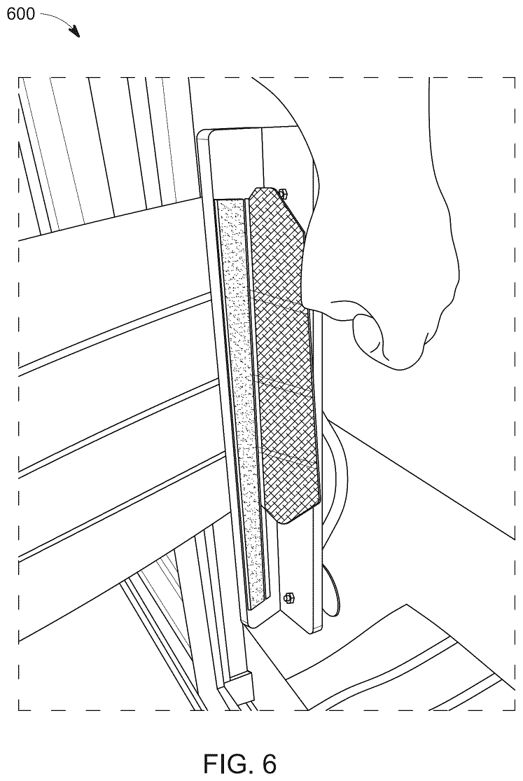

FIG. 6 shows a user's hand gripping the compression aid 100 mounted on a door. This figure demonstrates the accessibility and ease of use of the device, allowing users to manipulate the abdominal binder without assistance from others.

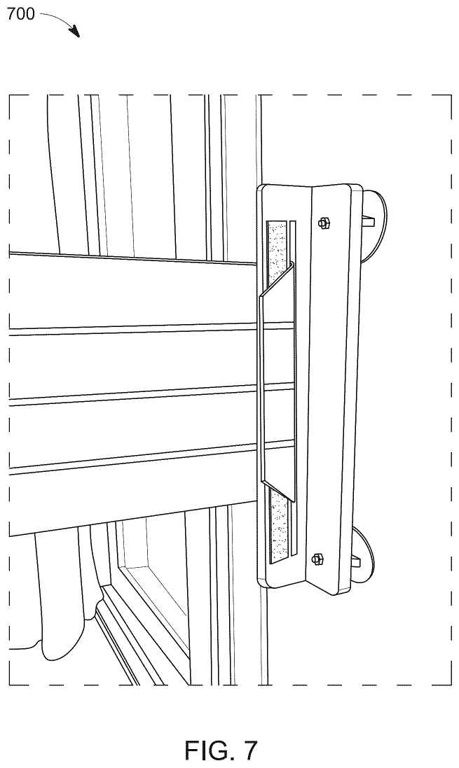

FIG. 7 illustrates the compression aid 100 mounted on a sliding door. This figure showcases the versatility of the device, as it can be attached to various types of doors or other suitable surfaces in a user's environment.

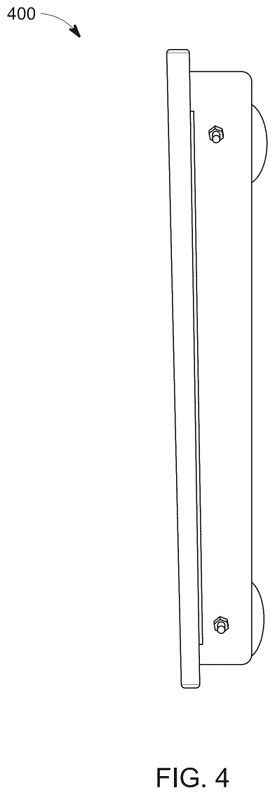

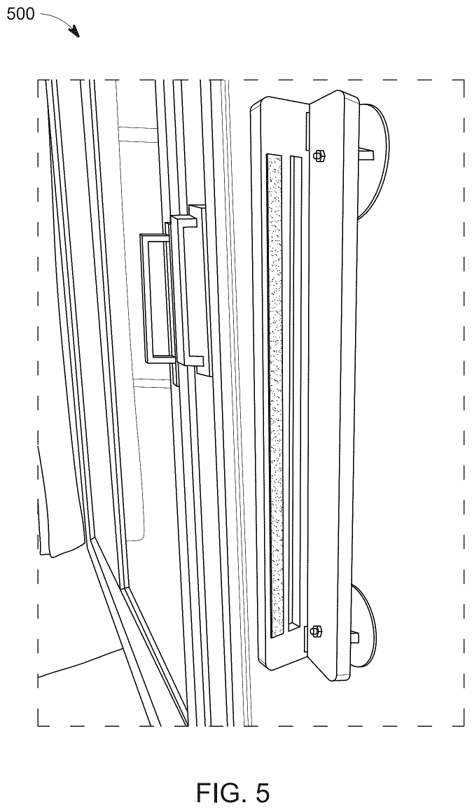

Referring now to FIG. 4 and FIG. 5, the compression aid 100 may be permanently mounted using screws. Holes 116 are provided on the compression aid 100 for this purpose. These holes 116 allow screws to pass through the compression aid 100 and into a wall stud or other suitable fixed structure. This mounting option may be particularly useful when a permanent installation is desired. The same holes 116 that accommodate screws for permanent installation are also used to attach the suction cups 112, illustrating the dual functionality of these features.

Turning to FIG. 1, the strap slots 114 may be positioned on the side of the compression aid 100 where the suction cups 112 are located. This arrangement is particularly useful when the compression aid 100 is created using a molding process. Alternatively, as depicted in FIG. 5, the strap slots 114 may also be located on the bottom of the top perpendicular piece of the compression aid 100. This configuration is suitable for manufacturing in a machine shop, demonstrating the adaptability of the compression aid 100 in terms of mounting and manufacturing processes. In one or embodiments, the mounting of the binder compression aid involves using suction cups to attach the compression aid 100 to a heavy solid glass surface.

In use, a method for self-applying an abdominal binder using the compression aid 100 may include the following steps:
1. Securely mounting the compression aid 100 to a fixed surface at an appropriate height for the user. This may involve using the suction cups 112 on a glass surface, or threading straps through the strap slots 114 to attach the device to a door frame or other structure.
2. Threading one end of the abdominal binder through the wrap pass thru slot 108.
3. Securing the end of the binder to the hook-and-loop fastener strip 110 on the protruding section 106 of the compression aid 100.
4. Stretching the binder out and beginning to wrap it around the body, maintaining tension as needed.
5. Rotating the body toward the compression aid 100 while continuing to wrap the binder.
6. Once fully wrapped, detaching the binder from the hook-and-loop fastener strip 110 on the compression aid 100.
7. Securing the final attachment of the binder around the body.

This method allows users to achieve proper tension and positioning of the abdominal binder without requiring assistance from another person. The compression aid 100 effectively acts as a “third hand,” holding one end of the binder securely while the user wraps it around their body.

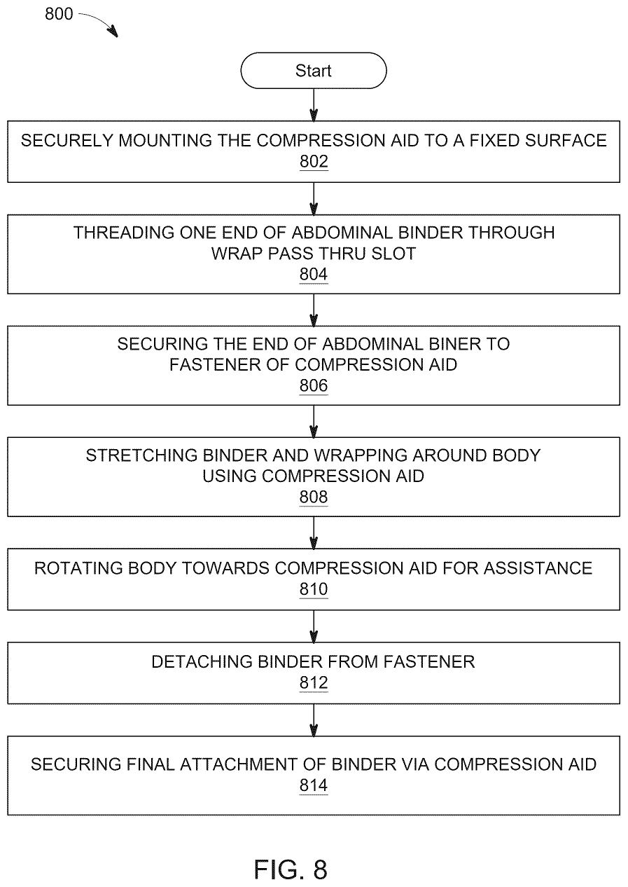

FIG. 8 is a flowchart illustrating an example method for implementing the binder compression aid according to various embodiments of the present disclosure. The method may include the following steps: Step 802: Securely mounting the compression aid 100 to a fixed surface at an appropriate height for the user. This step may involve using the suction cups 112 on a glass surface, or threading straps through the strap slots 114 to attach the device to a door frame or other structure. Step 804: Threading one end of the abdominal binder through the wrap pass thru slot 108 of the compression aid 100. Step 806: Securing the end of the binder to the hook-and-loop fastener strip 110 on the protruding section 106 of the compression aid 100. Step 808: Stretching the binder out and beginning to wrap it around the body, maintaining tension as needed. Step 810: Rotating the body toward the compression aid 100 while continuing to wrap the binder. Step 812: Once fully wrapped, detaching the binder from the hook-and-loop fastener strip 110 on the compression aid 100. Step 814: Securing the final attachment of the binder around the body. This method may allow users to achieve proper tension and positioning of the abdominal binder without requiring assistance from another person. The compression aid 100 may effectively act as a third hand, holding one end of the binder securely while the user wraps it around their body. The steps of the method may be performed in various orders or combinations. Some steps may be optional or performed multiple times. The specific implementation may vary based on the user's needs and preferences.

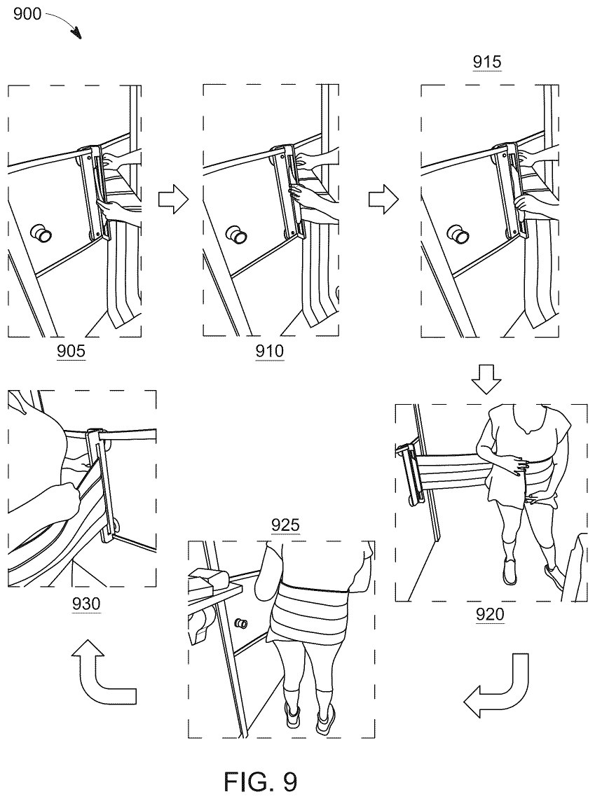

Referring to FIG. 9, 900 a method of using the binder compression aid utilizing a door frame is depicted. The compression aid may be secured to a door frame at a suitable height for the user at step 905. This may involve threading straps through the strap slots to attach the device to the door frame as shown in step 910. Once secured, one end of the abdominal binder may be threaded through the wrap pass thru slot of the compression aid as shown in step 915. The end of the binder may then be secured to the hook-and-loop fastener strip on the protruding section of the compression aid. The user may then stretch the binder out and begin to wrap it around their body, maintaining tension as needed as shown in step 920. The user may rotate their body toward the compression aid while continuing to wrap the binder as shown in step 925. Once fully wrapped, the binder may be detached from the hook-and-loop fastener strip on the compression aid as shown in step 930. The final attachment of the binder around the user's body may then be secured. This method allows the user to apply the abdominal binder independently, using the door frame as a stable structure for mounting the compression aid.

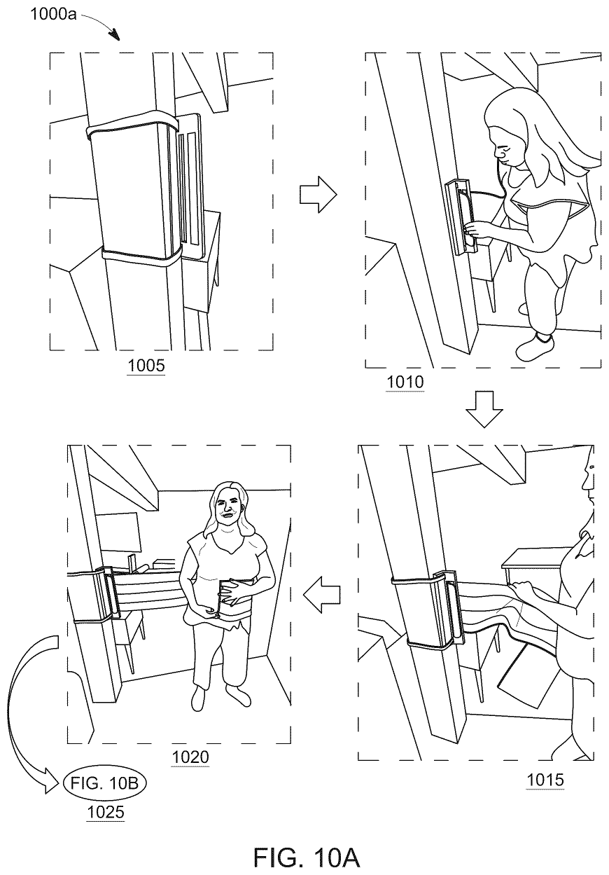

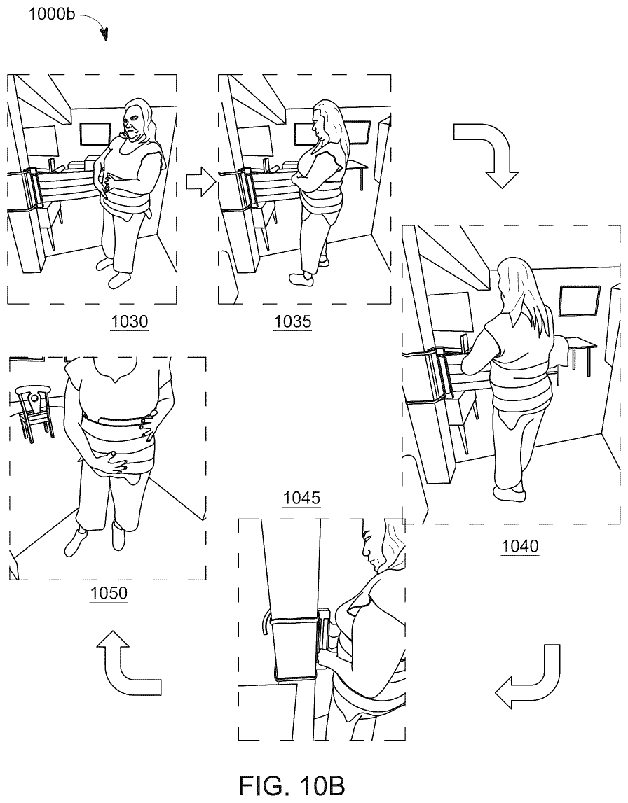

FIG. 10A and FIG. 10B illustrate a method of using the binder compression aid utilizing a post. In FIG. 10A, 1000A the compression aid may be attached to a post at an appropriate height as shown in step 1005. This may involve using straps threaded through the strap slots to securely mount the device as shown in step 1010. With the compression aid in place, the user may thread one end of the abdominal binder through the wrap pass thru slot. The binder end may be secured to the hook-and-loop fastener strip on the protruding section of the compression aid as shown in step 1015. The user may then stretch the binder out and begin to wrap it around their body, as shown in step 1020. The method may continue from FIG. 10A to FIG. 10B at step 1025. By maintaining tension and rotating their body toward the compression aid, the user can effectively wrap the binder. Wrapping the binder is shown in steps 1030, 1035 and step 1040. Once the wrapping is complete, the binder may be detached from the hook-and-loop fastener strip as shown in step 1045. The final attachment may be secured around the user's body as shown in step 1050. This method facilitates the self-application of the abdominal binder using a post as the support structure for the compression aid.

The compression aid 100 may be made from various materials, including but not limited to plastics, metals, or composite materials. The choice of material may depend on factors such as durability, weight, cost, and ease of cleaning. In some embodiments, the device may be made from materials suitable for sterilization, allowing for use in medical settings.

While the compression aid 100 has been described primarily in the context of post-surgical recovery, it should be understood that it may have applications in various other situations where abdominal compression or support is desired. For example, it may be useful for individuals with chronic back pain, during pregnancy, for general posture support, or help in body contouring. The compression aid 100 may be produced in various sizes to accommodate different user heights and body types. Additionally, the device may be adjustable in some embodiments, allowing for customization to individual needs.

In some embodiments, the compression aid 100 may include additional features such as padding on the mounting surface to protect doors or walls, or markers or guides to help users position the binder consistently each time it is applied. The compression aid 100 provides several advantages over traditional methods of applying abdominal binders. It promotes independence for users, potentially improving consistency of binder use during recovery. By allowing users to control the tension themselves, it may also lead to more comfortable and effective binder application.

It should be understood that the embodiments described herein are exemplary and that a person skilled in the art may make many variations and modifications without departing from the spirit and scope of the disclosure. All such variations and modifications are intended to be included within the scope of the disclosure as defined in the appended claims.

While illustrative embodiments of the invention have been shown and described, variations and alternative embodiments may occur to those skilled in the art. Such variations and alternative embodiments may be made without departing from the scope of the invention as defined in the claims.

As used in this specification and the appended claims, the singular forms “a” and “an” indicate a single element, while “the” may refer back to single or plural referents. Unless defined otherwise, all technical and scientific terms used herein have the same meaning as commonly understood by one of ordinary skill in the art to which the disclosure pertains.

The above detailed description of exemplary and preferred embodiments is presented for the purposes of illustration and disclosure in accordance with the requirements of the law. It is intended to be exemplary but not exhaustive, and is not intended to limit the invention to the precise forms described, but only to enable others skilled in the art to understand how the invention may be suited for a particular use of implementation. No limitation is intended by the description of exemplary embodiments which may have included tolerances, feature dimensions, specific operating conditions, engineering specifications, or the like, and which may vary between implementations or with changes to the state of the art, and no such limitation should be implied therefrom.

Applicant has made this disclosure with respect to the current state of the art, but also contemplates advancements and that adaptations in the future may take into consideration those advancements in accordance with the then current state of the art. It is intended that the scope of the invention be defined by the Claims as written and equivalents as applicable. Reference to a claim element in the singular is not intended to mean “one and only one” unless explicitly so stated. No claim element herein is intended to be construed under the provisions of 35 U.S.C. 112(f), unless the element is expressly recited using the exact phrase “means for . . . ” and no method or process step herein is to be construed under the provisions of 35 U.S.C. section 112(f) unless the step, or steps, are expressly recited using the exact phrase “step(s) for . . . ”.

While aspects of the present disclosure can be described and claimed in a particular statutory class, such as the system statutory class, this is for convenience only and one of skill in the art will understand that each aspect of the present disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way appreciably intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications can be referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present disclosure is not entitled to antedate such publication by virtue of prior present disclosure. Further, the dates of publication provided herein can be different from the actual publication dates, which can require independent confirmation.

The patentable scope of the present disclosure is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims.

Insofar as the description above and the accompanying drawing disclose any additional subject matter that is not within the scope of the claims below, the disclosures are not dedicated to the public and the right to file one or more applications to claims such additional disclosures is reserved.

The foregoing descriptions of specific embodiments of the present invention have been presented for purposes of illustration and description. They are not intended to be exhaustive or to limit the present invention to the precise forms disclosed, and modifications and variations are possible in view of the above teaching. The exemplary embodiment was chosen and described to best explain the principles of the present invention and its practical application, to thereby enable others skilled in the art to best utilize the present invention and its embodiments with modifications as suited to the use contemplated.

It is therefore submitted that the present invention has been shown and described in the most practical and exemplary embodiments. It should be recognized that departures may be made which fall within the scope of the invention. With respect to the description provided herein, it is submitted that the optimal features of the invention include variations in size, materials, shape, form, function and manner of operation, assembly, and use. All structures, functions, and relationships equivalent or essentially equivalent to those disclosed are intended to be encompassed by the present invention.

It should be understood that the above-described embodiments are illustrative of only a few of the possible specific embodiments which can represent applications of the principles of the present disclosure. Numerous and varied other arrangements can be readily devised by those skilled in the art without departing from the spirit and scope of the disclosure. While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. A binder compression aid comprising:
a bracket configured to be mounted to a fixed surface, the bracket comprising:
a mounting section for attaching to the fixed surface;
a protruding section extending from the mounting section;
a slot in the protruding section configured to receive an abdominal binder; and
a securing mechanism on the protruding section configured to temporarily hold one end of the abdominal binder.

2. The binder compression aid of claim 1, wherein the securing mechanism comprises a strip of hook-and-loop fastener material.

3. The binder compression aid of claim 1, further comprising at least one attachment mechanism for mounting the bracket to the fixed surface, wherein the fixed surface is a non-porous surface.

4. The binder compression aid of claim 3, wherein the at least one attachment mechanism comprises suction cups.

5. The binder compression aid of claim 3, wherein the at least one attachment mechanism comprises strap slots configured to receive straps for securing the bracket to the fixed surface.

6. The binder compression aid of claim 1, wherein the mounting section and the protruding section are perpendicular to each other.

7. The binder compression aid of claim 1, wherein the slot is approximately 16.5 inches long and 0.5 inches wide.

8. The binder compression aid of claim 1, wherein the mounting section and the protruding section are each approximately 15 to 20 inches long and 2.5 to 3 inches wide.

9. A method for self-applying an abdominal binder, the method comprising:
mounting a binder compression aid to a fixed surface, the compression aid comprising a bracket with a slot and a securing mechanism;
threading one end of an abdominal binder through the slot in the bracket;
securing the one end of the abdominal binder to the securing mechanism on the bracket;
stretching the abdominal binder and wrapping it around a user's body while maintaining tension;
rotating the user's body toward the compression aid while continuing to wrap the abdominal binder; and
detaching the abdominal binder from the securing mechanism on the bracket and completing the attachment of the abdominal binder around the user's body.

10. The method of claim 9, wherein mounting the binder compression aid comprises attaching the compression aid to a glass surface using suction cups.

11. The method of claim 9, wherein mounting the binder compression aid comprises securing the compression aid to a door frame using straps.

12. The method of claim 9, wherein securing the one end of the abdominal binder to the securing mechanism comprises attaching the abdominal binder to a strip of hook-and-loop fastener material on the bracket.

13. The method of claim 9, further comprising adjusting the tension of the abdominal binder by varying the distance between the user's body and the compression aid during the wrapping process.

14. An abdominal binder application system comprising:
an abdominal binder; and
a compression aid configured to assist in self-application of the abdominal binder, the compression aid comprising:
a bracket having a mounting section and a protruding section;
a slot in the protruding section sized to receive the abdominal binder;
a securing mechanism on the protruding section configured to temporarily hold one end of the abdominal binder; and
at least one attachment mechanism for mounting the bracket to a fixed surface.

15. The system of claim 14, wherein the securing mechanism comprises a strip of hook-and-loop fastener material.

16. The system of claim 14, wherein the at least one attachment mechanism comprises suction cups for mounting the bracket to a glass surface.

17. The system of claim 14, wherein the at least one attachment mechanism comprises strap slots configured to receive straps for securing the bracket to a door frame.

18. The system of claim 14, wherein the abdominal binder comprises a strip of hook-and-loop fastener material at one end for securing the binder around a user's body.

19. The system of claim 14, wherein the mounting section and the protruding section of the bracket are perpendicular to each other.

20. The system of claim 14, wherein the slot in the protruding section is approximately 16.5 inches long and 0.5 inches wide, and the mounting section and the protruding section are each approximately 15 to 20 inches long and 2.5 to 3 inches wide.
